(19) United States
(12) Patent Application Publication
(10) Pub. No.: US 2024/0000503 A1
(43) Pub. Date: Jan. 4, 2024

(54) SINGLE-PIECE UTERINE CAVITY ELECTRIC RESECTION DEVICE

(71) Applicant: Chongyi ZHANG, Xi'an, Shaanxi (CN)

(72) Inventor: Chongyi ZHANG, Xi'an, Shaanxi (CN)

(21) Appl. No.: 17/919,634

(22) PCT Filed: Dec. 9, 2020
(86) PCT No.: PCT/CN2020/134858

§ 371 Date: Sep. 5, 2023

(30) Foreign Application Priority Data
Apr. 23, 2020 (CN) 202020627002.4
Jul. 6, 2020 (CN) 202010642586.7
Jul. 6, 2020 (CN) 202021302233.4

(51) Int. Cl. A61B 18/14

(52) U.S. Cl. CPC A61B 18/1482; A61B 2018/00559

(57) ABSTRACT

The present application discloses a single-piece uterine cavity electric resection device, which includes a fixing rod, an operating handle, a traction wire, and a cutting electrode. The traction wire is driven to move along the guide sliding sleeve on the fixing rod by the opening and closing the operating handle, so as to control the support body connected with the traction wire, and drive the cutting electrode to unfold, fold and change the opening angle under the limitation of pulling back or pulling of the tension wire. When entering and exiting the uterine cavity, the cutting electrode can be folded under the operating of the handle, which can make the electric resection device enter and exit the uterine cavity easily, and the cutting electrode is not easy to be damaged. During treatment, the angle of cutting electrode can be adjusted flexibly and accurately in a larger range, the treatment effect is better, and the operation process is safer. The present application can not only reduce the treatment cost and time, but also reduce the degree of treatment pain, and provides convenient conditions for completing corresponding surgical operations in the outpatient department.

### TECHNICAL FIELD

The present application relates to electric resection apparatuses of a hysteroscopic surgical treatment system, and more particularly to a single-piece uterine cavity electric resection ring and a single-piece uterine cavity electric resection needle that can be separated from a hysteroscope.

### BACKGROUND

In resectoscopes, electric resection apparatuses such as electric resection rings and electric resection needles are usually packaged together with the endoscope, and the cutting electrodes and operating rods of electric resection rings and electric resection needles are fixed vertically to each other. During intrauterine treatment, in order to avoid the damage of the cutting electrodes and other parts (for example, when entering and exiting the external cervical orifice, cervical canal and internal orifice), the electric resection rings and electric resection needles need to be retracted into the inside of the resectoscope. Therefore, the diameter of the resectoscope is larger, which results in that the uterine orifice needs to be expanded to 8-11 mm when in used, and the greater the uterine orifice expanded, the greater damage to the cervix, the greater the possibility of cervical insufficiency caused after surgery. Furthermore, the resectoscope has a fine structure, and the price of the resectoscope is higher, which is easy to be damaged during operation, such that the hysteroscopic surgery in outpatient departments has many constraints.

Due to fact that the electric resection ring and electric resection needle in the resectoscope are fixed with the operating rod, and the angle cannot be adjusted independently during the electric resection treatment, which leads to the electric resection treatment is complex and the time of electric resection treatment is increased with the development of lesions (for example, the larger the submucosal fibroids) when polyps, intrauterine adhesions, and submucosal fibroids are found in the uterine cavity during hysteroscopy. Further, the incidence of hyponatremia and water poisoning increased significantly, and the difficulty of hysteroscopic surgery in the outpatient department is increased.

Chinese patent CN208426206U disclosed a foldable electrode resectoscope, in which the electric resection apparatus uses a spring to connect the cutting electrode and the metal sleeve, so that the cutting electrode can be rotated at a certain angle, and the resecting is completed by pushing and pulling, which improves the surgical efficiency of decompression of renal cyst. However, the electric resection apparatuses and the endoscope of the Chinese patent CN208426206U are still in an integrated structure, and the resectoscope is large in size. Furthermore, the spring part is a conductor and is connected to the cutting electrode as a whole, and also used for resecting. Therefore, the electrode resectoscope needs a large size of uterine orifice expanded when the electrode resectoscope is used in intrauterine surgery, and it is easy to damage the normal tissue next to the lesion during operation, which increases the risk of complications.

At present, there are no reports of a single-piece uterine cavity electric resection device used in conjunction with an independent hysteroscope.

### SUMMARY

### Technical Problem

One of objects of the present application is to provide a single-piece uterine cavity electric resection device.

In order to solve above-mentioned object, the technical solution adopted in the present application as following:

The single-piece uterine cavity electric resection device includes: a fixing rod, an operating handle, a traction wire, and a cutting electrode. The fixing rod is provided with a guide sliding sleeve and a support body that can swing to any end (for example, a front end and rear end) of the fixing rod, the operating handle is arranged at one end (for example, the rear end) of the fixing rod, and two ends of the traction wire are respectively extended along a turn-back channel formed by the guide sliding sleeve at the other end (for example, the front end) of the fixing rod, and connected to the operating handle and the support body, a tensioning wire extending outward and connected to the support body is arranged in the fixing rod; the cutting electrode is arranged on the support body, and the cutting electrode is connected with a cable extending along the fixing rod.

Optionally, the guide sliding sleeve includes a bending tube body and a straight tube body, an inner concave side of the bending tube body is covered on one end (for example, the front end) of the fixing rod, and the straight tube body is extended to the other end (for example, the rear end) of the fixing rod where the operating handle is arranged.

Optionally, the support body is in a shape of an arc-shaped shell, one end of the support body is connected to the fixing rod by a pin, and the other end of the support body is connected to the cutting electrode configured to be a ring electrode; one end of the cable is connected to an extraction electrode arranged inside the fixing rod, or arranged on an outer surface of the fixing rod, a main body of the cable is arranged inside the fixing rod, and the other end of the cable is extended to a position where the ring electrode is connected with the support body.

Optionally, the support body is in a shape of a hollow cylinder, one end of the support body is connected to the fixing rod by a pin, and the other end of the support body is connected to the cutting electrode configured to be a needle electrode; one end of the cable is connected to an extraction electrode arranged inside the fixing rod or arranged on an outer surface of the fixing rod, a main body of the cable is arranged inside the fixing rod, and the other end of the cable is extended onto the support body.

Optionally, the operating handle includes a pair of handle bodies that are mutually hinged, the tensioning wire is connected with one of the pair of handle bodies engaged on a corresponding end (for example, the rear end) of the fixing rod, and the other one of the pair of handle bodies is connected with the traction wire.

Optionally, a cross-sectional dimension of the fixing rod is gradually decreased from one end (for example, the rear end) of the fixing rod provided with the operating handle to the other end (for example, the front end) of the fixing rod.

Optionally, a swing angle of the support body is ranged from 30 degree to 120 degree.

Optionally, a diameter of the cutting electrode is ranged from 0.5 mm to 1.5 mm.

The beneficial effects of the present application are as follows:

In the present application, electric resection device of the present application can drive the traction wire move along the guide sliding sleeve on the fixing rod by opening and closing the operating handle, so as to control the support body connected with the traction wire, and drive the cutting electrode (such as needle electrode and ring electrode) to unfold, fold and change the opening angle under the limitation of pulling back or pulling of the tension wire. The cutting electrode can be retracted when entering and exiting the uterine cavity, which can make the electric resection device enter and exit the uterine cavity easily without expanding the uterine orifice or reducing the size of expanding the uterine orifice, and the cutting electrode is not easy to be damaged. In hysteroscopic treatment, a hysteroscope and the electric resection device of the present application can be hold respectively and cooperated with each other to complete the operation, which can not only continuously complete the operation of removing lesions (polyps, intrauterine adhesions, submucosal fibroids) at different angles and depths (with greater scope and flexibility) in a certain field of vision by the doctors, so as to make the treatment process simple and achieves better therapeutic effect, but also the operation process is safer. Furthermore, the popularization and application of the present application can also reduce the cost of the apparatuses (the price of the existing integrated hysteroscope is much higher than the price of the single-piece uterine cavity electric resection device), the treatment cost and time, reduce the degree of treatment pain, and complete the corresponding surgical operations in the outpatient department.

Furthermore, when the present application is applied to hysteroscopy and vaginal endoscopy, according to the size of the lesion, different opening angles of the cutting electrode (for example, 45-120 degrees) are formed by operating the swing angle of the support body, which can not only perform the electric resection treatment for polyps, intrauterine adhesions, submucosal fibroids, etc., but also provide effective electric coagulation for small blood vessels in the uterine cavity.

Furthermore, the present application can maximize the tissue cutting by using a cutting electrode with a larger diameter, thereby shortening the operation time and reducing the risk of complications such as water poisoning.

Furthermore, in the present application, the operating handle of the electric resection device is located at the thicker rear end of fixing rod, and the fixing rod gradually tapers towards the front end. In the treatment, the front end of the electric resection device is easier to enter (for example, along the uterine cavity to check the scope body) and pass through the narrower part of the uterine cavity (for example, the internal opening of the cervix), so as to improve the operation efficiency.

Furthermore, the support body is arranged in a shape of an arc-shaped shell or in a shape of a hollow cylinder, which in combination with flexible angle adjustment, the electric resection of non-cutting electrodes on tissues can be effectively reduced, and the injuring and damage to normal tissues caused by misoperation in the electric resection can be avoided.

### BRIEF DESCRIPTION OF THE DRAWINGS

FIG. 1 shows a front view of a single-piece electric resection ring in Embodiment 1, in which, a ring electrode is folded to 30 degrees;

FIG. 2 is a schematic view of the connection between a support body and a ring electrode in Embodiment 1, where the fixing rod is shown in side view;

FIG. 3 is a schematic view of cable connection in Embodiment 1;

FIG. 4 shows a front view of a single-piece electric resection ring in Embodiment 1, in which, a ring electrode is unfolded to 90 degrees;

FIG. 5 is a schematic view of the connection between the support body and the ring electrode in Embodiment 1, where the fixing rod is shown in top view;

FIG. 6A is a front view of a single-piece electric resection ring in Embodiment 1, in which, a ring electrode is unfolded to 120 degrees, and a tensioning wire in a fixing rod is not shown;

FIG. 6B is a front view of a single-piece electric resection ring in Embodiment 1, in which, a ring electrode is unfolded to 120 degrees, and a tensioning wire in a fixing rod is shown;

FIG. 7 shows a front view of a single-piece electric resection needle in Embodiment 2, in which the needle electrode is folded to 30 degrees;

FIG. 8 shows a front view of a single-piece electric resection needle in Embodiment 2, in which the needle electrode is unfolded to 90 degrees;

FIG. 9 is a first schematic view of the connection between the support body and the needle electrode in Embodiment 2;

FIG. 10 is a second schematic view of the connection between the support body and the needle electrode in Embodiment 2;

FIG. 11 shows a schematic view of cable connection in Embodiment 2; and

FIG. 12 shows a front view of a single-piece electric resection needle in Embodiment 2, in which, a ring electrode is unfolded to 120 degrees.

In the drawings, reference signs are listed as following:
1-1 first handle body; 1-2 second handle body; 1-3 handle connection portion; 1-4 traction wire; 1-5 fixing rod; 1-6 tensioning wire; 2 guide sliding sleeve; 3 support body; 3-1 movable connection point; 3-2 fixed connection point; 4 ring electrode; 5 cable; 6 extraction electrode; and 7 needle electrode.

### DETAILED DESCRIPTION OF EMBODIMENTS

The present application will be further described in detail below in combination with the drawings and embodiments.

At present, all the hysteroscopes used are cylindrical, with a diameter of more than 8 mm, which are developed from the male prostate resectoscope. However, the structure of the male urethral system is quite different from that of the female vaginal and uterine reproductive systems. In order to make good use of the physiological structure of women themselves, the present application separates the uterine cavity examination system of the resectoscope from the electric resection system, and improves the structure of the electric resection system, thus a single-piece electric resection system is formed. The cutting electrode of which can effectively avoid rubbing with tissues when entering and leaving the uterine cavity, and can play a therapeutic role in a wider range with the cooperation of the inspectoscope, so as to make better use of the physiological structure of women themselves for treatment, which can reduce the difficulty and cost of treatment, and further reduce the pain of patients.

### Embodiment 1

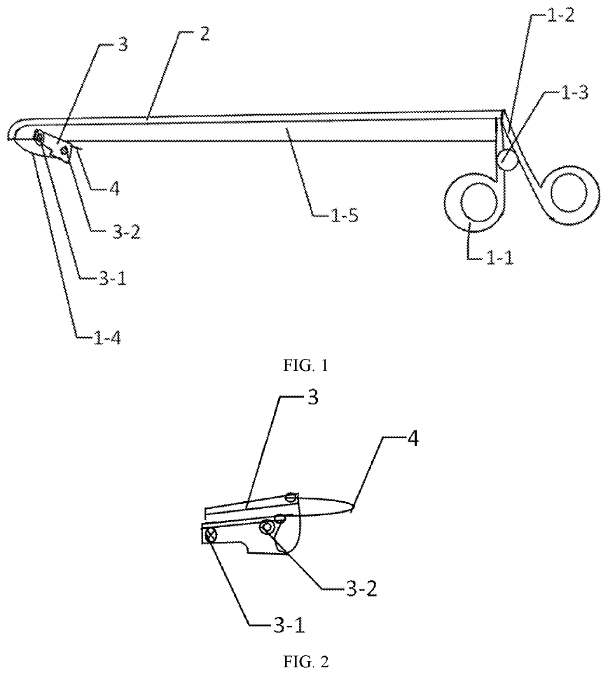

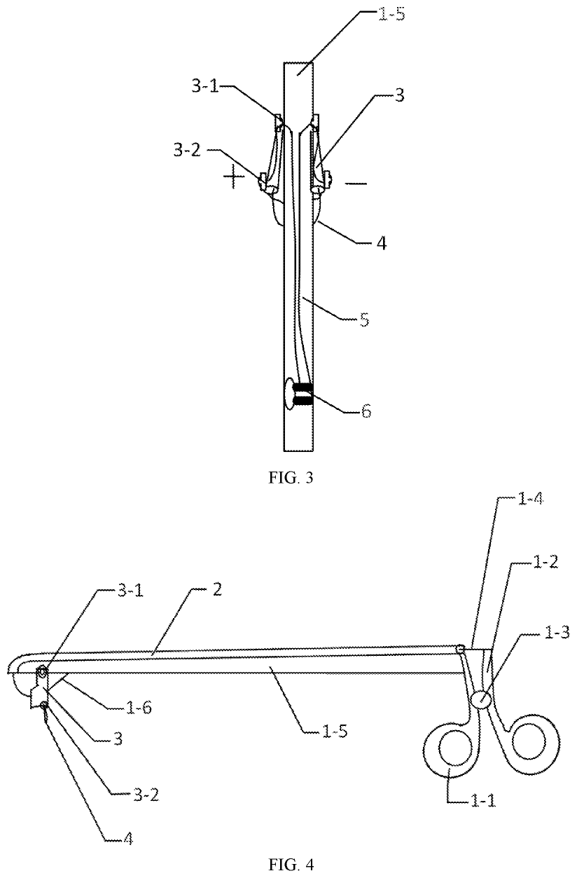

As shown in FIG. 1, FIG. 3, and FIG. 6B, the single-piece electric resection ring provided in the embodiment includes a handle, a fixing rod 1-5, a plurality of flexible wires with certain strength that can be bent freely (for example, synthetic fibers such as nylon, polymer such as PE, metal alloy materials such as titanium steel) and a circuit loop system. The handle includes a first handle 1-1 and a second handle 1-2 which are connected (hinged) through the handle connection portion 1-3. The first handle 1-1 is clamped and fixed with the rear end of the fixing rod 1-5. The second handle 1-2 is connected with a section of the above flexible wire and acts as a traction wire 1-4. The traction wire 1-4 passes through the guide sliding sleeve 2 which is integrally located on the upper part of the fixing rod 1-5. The traction wire 1-4 is horizontally led out from the second handle 1-2 and is extended toward the front end of the fixing rod 1-5. The front end of the traction wire 1-4 passes through a bending section (a section of bending tube body) of the guide sliding sleeve 2, and the bending section is closed to the outside of the front end of the fixing rod 1-5, so that the traction wire 1-4 can turn back a certain distance to the rear end of the fixing rod 1-5 by bending (along the bent tube body), and finally connected with the movable support body 3 (arc-shaped shell) connected close to the front end of the fixing rod 1-5. The another section of the above flexible wire extends from the inside of the fixing rod 1-5 along the direction toward the front end of the fixing rod 1-5, and is connected with the support body 3. The specific connection position can be selected at the back side of the connection position between the traction wire 1-4 and the support body 3. The section of flexible wire passes through one end of the fixing rod 1-5 and reaches the rear end of the fixing rod 1-5 along the inside of the fixing rod 1-5, and is connected with a spring preset inside the fixing rod 1-5. Thus a pulling back tension wire 1-6 is formed. If the preset spring is not arranged, the flexible wire can be retracted by using the wire winding mechanism (for example, a fixed knob) arranged on the fixing rod 1-5 or the first handle 1-1 to form a traction tensioning wire. The circuit loop system includes a ring electrode 4 installed on the support body 3 (arc-shaped shell), a cable 5 with a main body being embedded in the fixing rod 1-5, and positive and negative extraction electrodes 6. The extraction electrodes 6 are connected with the external control circuit through the wiring hole on the fixing rod 1-5. One end of the cable 5 is respectively connected with the positive and negative extraction electrodes 6, and the other end is led out of the fixing rod 1-5 and connected with both ends of the ring electrode 4.

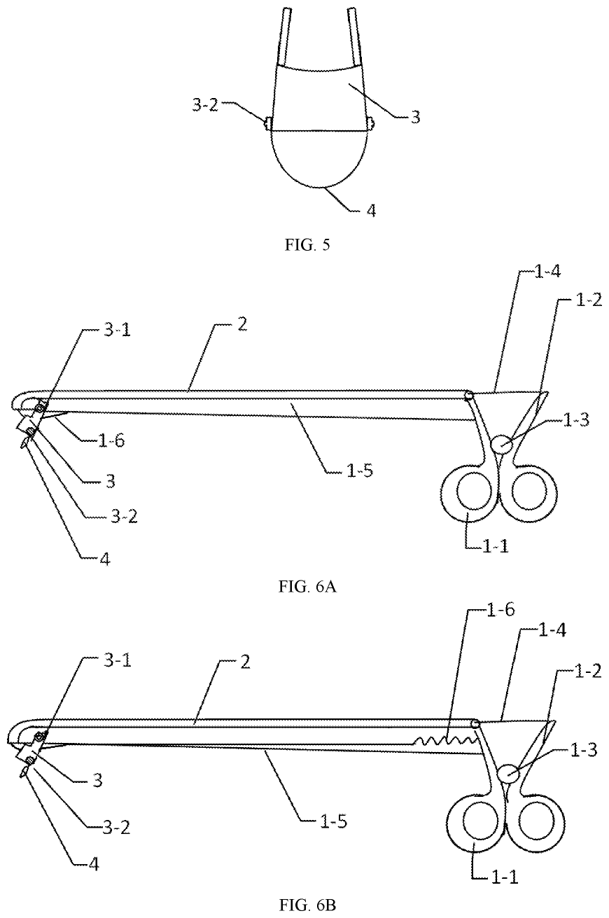

As shown in FIG. 1, FIG. 2, and FIG. 5, the support body 3 is machined from an arc-shaped shell intercepted from a side of a round table. The upper part of the arc-shaped shell only retains the part on both sides, the part on both sides are connected with the fixing rod 1-5 through pins respectively. The fixing rod 1-5 gradually widens along the radial section from the front end to the rear end. When the inner concave side of the arc-shaped shell turns to the fixing rod 1-5 with the pin (movable connection point 3-1) as the axis, the ring electrode 4 can be folded. In the folded state, the inner concave surface of the support body 3 (arc-shaped shell) is close to the fixing rod 1-5, so that the radial size of the electric resection ring (especially the part of the front end that needs to extend into the uterine cavity) reaches the minimum, and it is easy to pass through the external opening of the cervix and the internal opening of the uterine cavity, which solves the problem that the electric resection ring is easy to be damaged when passing through the cervical tube and the internal opening of the cervical tube. The lower part of the arc-shaped shell 1 is provided with an installation hole (fixed connection point 3-2), and the end portion of the ring electrode 4 can be fastened by screws, which is convenient for installing ring electrodes 4 of different sizes (thinner ring electrodes are used when electric resection of intrauterine adhesions; thicker ring electrodes are used when electric resection of leiomyomas), and also convenient for replacing ring electrodes 4 after resection consumption. The cable 5 can pass through the fixing rod 1-5 by the movable connection point 3-1 (for example, inside the pin).

As shown in FIG. 4 and FIG. 6A, with the opening of the handle, the second handle 1-2 pulls the traction wire 1-4, so that the support body 3 (arc-shaped shell) drives the ring electrode 4 to rotate towards the front end of the fixing rod 1-5. Furthermore, the tensioning wire 1-6 is pulled back. When the handle is fully open, the rotation angle of the ring electrode 4 (relative to the fixing rod) is 120 degrees. In the above rotation process, once the handle stops opening, the support body 3 (arc-shaped shell) and the ring electrode 4 are fixed to a position by the reverse traction of the traction wire 1-4 and the pulling back tensioning wire 1-6. According to the different therapeutic objectives in the uterine cavity, not only 90 degrees can be used, but also other different angles (for example, any angle between 45-120 degrees) can be selected for treatment. With the closing of the handle, the tension wire 1-6 is pulled back such that the support body 3 drives the ring electrode 4 to rotate towards the rear end of the fixing rod 1-5 until the support body 3 is abutted against the fixing rod 1-5. Due that the fixing rod 1-5 with gradually changing radial dimensions is applied, the pulling back tension wire 1-6 can be installed at an inclined angle, thus effectively reducing the extension length of the pulling back tension wire 1-6, and reducing the angle of the extension part as much as possible, thus reducing the interference to the operation.

The method of using above-mentioned single-piece electric resection ring:

As an independent electric resection device, the hysteroscopy can first be used to perform the inspection. When the electric resection treatment is needed, one of the methods is to use a vaginal speculum to expand the uterus properly according to the tightness of the uterine orifice. Generally, a diameter of the uterine orifice is required to be about 6 mm. According to the position of the uterus examined by hysteroscope, the single-piece electric resection ring is first sent into the uterine cavity, and then the single-piece electric resection ring is fixed with one hand, and the other one hand holds the hysteroscope and enters the uterine cavity from one side of the single-piece electric resection ring. The other method is not to use a vaginal speculum. Under the vaginal endoscope technology, the hysteroscopy is first be used to perform the inspection. When the electric resection treatment is needed, the hysteroscope is operated by one hand, the single-piece electric resection ring is hold by another hand, and the single-piece electric resection ring is first sent to the neck canal near the inner opening of the uterine cavity along the hysteroscope, then the hysteroscope is withdrawn back to the neck canal, and then the single-piece electric resection ring is sent into the uterine cavity under the direct vision of the hysteroscope, and then the hysteroscope is sent into the uterine cavity from one side of the single-piece electric resection ring. If the uterine orifice is relatively loose, the single-piece electric resection ring can also directly sent into the uterine cavity along the hysteroscope.

When the lesions requiring electric resection such as intrauterine polyps, intrauterine adhesions, and submucosal fibroids are detected, the single-piece electric resection ring is operated under hysteroscope for treatment. The handle is hold tightly to open the ring electrode 4, and different opening angles of the handle can be selected according to the required cutting position and depth. When electrification treatment is needed, stepping on the electrode plate to make the current flow into the ring electrode 4 through the extraction electrodes 6 and cable 5. For the single-piece electric resection ring (using the pulling back tension wire 1-6), except that the fixing rod 1-5 can be used to pull back or push forward, the angles of the ring electrode 4 can also be adjusted by opening or closing the handle, that is, the polyps, leiomyomas and other lesions can be removed by rotating the ring electrode 4, which is more in line with the principle of hoe weeding, so that the lesions can be completely removed to achieve better therapeutic effect. When necessary, the energized ring electrode 4 can also be used for coagulation during resection.

### Embodiment 2

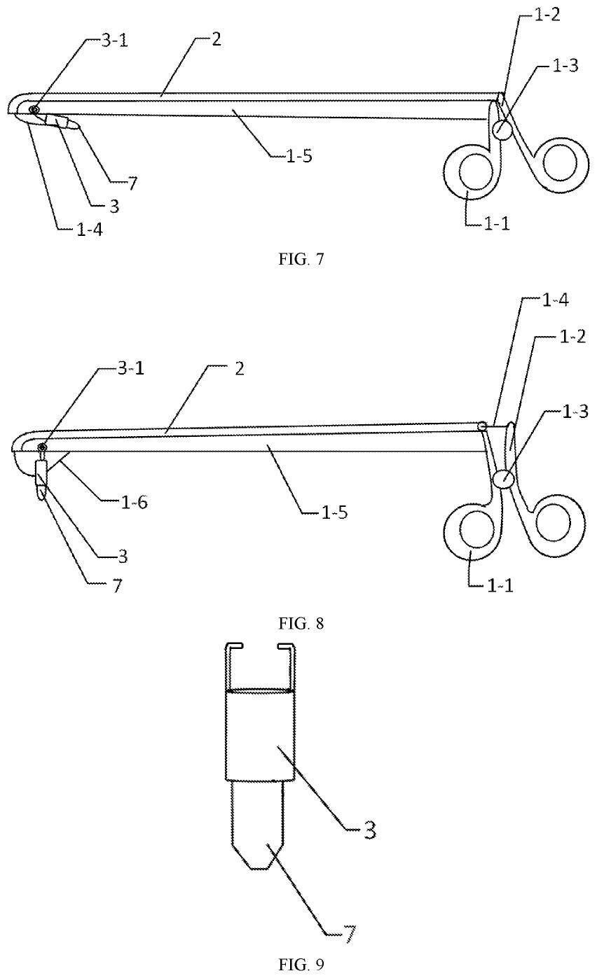

As shown in FIG. 7 and FIG. 11, the single-piece electric resection needle provided in the embodiment includes a handle, a fixing rod 1-5, a plurality of flexible wires with certain strength that can be bent freely (for example, synthetic fibers such as nylon, polymer such as PE, metal alloy materials such as titanium steel) and a circuit loop system. The different from Embodiment 1 are that the circuit loop system uses needle electrode 7 as the cutting electrode, and the support body 3 for installing needle electrode 7 is in the form of cylinder seat, which is convenient for installing needle electrode 7 and connecting with cable 5 leading from fixing rod 1-5. When the handle is closed, the support body 3 (in the form of cylinder seat) and needle electrode 7 are close to the fixing rod 1-5, and the support body 3 (in the form of cylinder seat) and needle electrode 7 have smaller radial dimensions compared with the fixing rod 1-5. Therefore, the overall radial dimension of the electric resection needle (except the handle) is minimized, and it is easy to pass through the external orifice of the cervix and the intrauterine orifice, which can avoid the contact damage of the needle electrode 7.

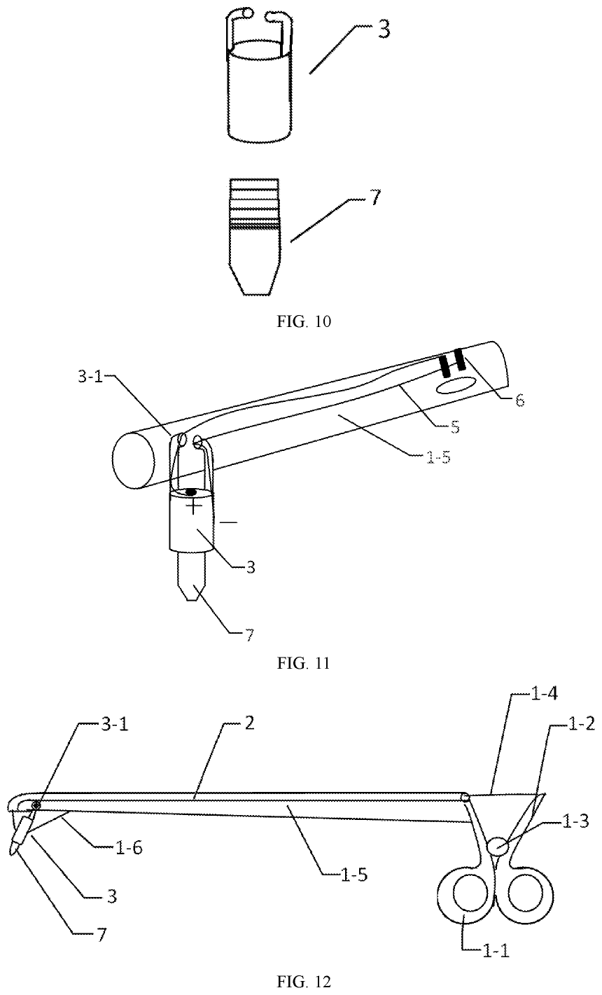

As shown in FIG. 9 and FIG. 10, the support body 3 (in the form of cylinder seat) and the fixing rod 1-5 are connected by pins. The different from Embodiment 1 are that the support body 3 (in the form of cylinder seat) includes a cylinder with internal threads. The lower part of the cylinder is connected and fixed with the tail end of the needle electrode 7 through the threads, the top part of the cylinder (the cable connected with one extraction electrode is connected on the top part of the cylinder, and the cable connected with the other extraction electrode is connected on the side wall of the cylinder) is installed with two inverted L-shaped pins. When the cylinder turns to the fixing rod 1-5 with the pins as the axis (movable connection point 3-1) the needle electrode 7 can be folded.

As shown in FIG. 8 and FIG. 12, with the opening or closing of the handle, the traction wire 1-4 is moved, and the angle of the needle electrode 7 relative to the fixing rod 1-5 is adjusted under the constraint of the force of pulling back tension wire 1-6. According to the degree of uterine cavity adhesion and the size of submucous myoma protruding to the uterine cavity, different angles can be selected for electric resection treatment between 60 and 120 degrees.

The use method of the above single-piece electric resection needle is basically the same as that of the single-piece electric resection ring in Embodiment 1, that is, the angle of the needle electrode 7 is adjusted by opening or closing the handle, and the needle electrode 7 is used for electric resection treatment of uterine cavity adhesions, submucosal myomas and other lesions under the energized state, which is more in line with the principle of electric resection treatment from shallow to deep, so as to achieve better therapeutic effect. When necessary, the energized needle electrode 7 can also be used for coagulation during resection.

When the electric resection device (electric resection ring or electric resection needle) in the above embodiments are used with the hysteroscope for treatment, the uterine orifice is pulled into an oval shape. The cross-sectional area of a hysteroscope with a diameter of 5 mm is 6.25π, which plus an electric resection device with a diameter of 4 mm and a cross-sectional area of 4π, the combined cross-sectional area of the two instruments is 10.25π, while the cross-sectional area of the uterine orifice with a diameter of 6 mm is 9π, and the cross-sectional area of the uterine orifice with a diameter of 7 mm is 12.25π. For most patients, the uterine orifice is about 6 mm, so there is no need to expand the uterine orifice. The smaller the expansion of the uterine orifice is, or even no uterine orifice expansion is required, then the damage to the cervix of the patient can be reduced to the minimum. Moreover, the operation of the hysteroscope and the electric resection device in the above embodiments is completed without the restriction of the speculum and cylindrical metal wrapping, so that the vision is wider and the range of activity is larger.

The single-piece electric resection ring and electric resection needle provided by the present application have the following characteristics:
1) The single-piece electric resection ring and the electric resection needle of the present application are more consistent with the physiological structure of human uterine cavity. In the treatment with hysteroscope (inspectoscope), the single-piece electric resection ring and the electric resection needle are smaller in the front end when they are folded, with a diameter of about 4 mm, and can be fed separately. Therefore, the size of uterine orifice expansion is reduced, the damage to the cervix is less, and the complications caused are less.
2) The single-piece electric resection ring and the electric resection needle of the present application have a wider range of activities in the intrauterine treatment, which improves the electric resection effect by changing the cutting electrode (such as a thicker diameter), correspondingly speed up the operation process and reduce the risk of complications.
3) The single-piece electric resection ring and electric resection needle of the present application can adjust the angle of the cutting electrode in a larger range, which solves the problem that the operation of the existing uterine cavity resectoscope is limited by the position of the uterus, the size of the inner opening and the speculum, and can increase the tissues for each resection at any time according to the needs, thereby significantly shortening the electric resection operation time and reducing the surgical complications.
4) The single-piece electric resection ring and the electric resection needle of the present application basically do not need to expand the uterine orifice, and the injury is small, so the treatment process can be completed in the outpatient operating room, which not only reduces the treatment cost, but also can achieve good treatment effect.

1. A single-piece uterine cavity electric resection device, comprising:
a fixing rod (1-5), provided with a guide sliding sleeve (2) and a support body (3) that can swing to any end of the fixing rod (1-5), wherein a tensioning wire (1-6) extending outward and connected to the support body (3) is arranged in the fixing rod (1-5);
an operating handle, arranged at one end of the fixing rod (1-5);
a traction wire (1-4), two ends of which respectively extended along a turn-back channel formed by the guide sliding sleeve (2) at the other end of the fixing rod (1-5), and connected to the operating handle and the support body (3); and
a cutting electrode, arranged on the support body (3), wherein the cutting electrode is connected with a cable (5) extending along the fixing rod (1-5).

2. The single-piece uterine cavity electric resection device according to claim 1, wherein the guide sliding sleeve (2) comprises a bending tube body and a straight tube body, an inner concave side of the bending tube body is covered on one end of the fixing rod (1-5), and the straight tube body is extended to the other end of the fixing rod (1-5) where the operating handle is arranged.

3. The single-piece uterine cavity electric resection device according to claim 1, wherein the support body (3) is in a shape of an arc-shaped shell, one end of the support body (3) is connected to the fixing rod (1-5) by a pin, and the other end of the support body (3) is connected to the cutting electrode.

4. The single-piece uterine cavity electric resection device according to claim 3, wherein the cutting electrode is configured to be a ring electrode (4), one end of the cable (5) is connected to an extraction electrode arranged inside the fixing rod (1-5) or arranged on an outer surface of the fixing rod, a main body of the cable (5) is arranged inside the fixing rod (1-5), and the other end of the cable (5) is extended to a position where the ring electrode (4) is connected with the support body (3).

5. The single-piece uterine cavity electric resection device according to claim 1, wherein the support body (3) is in a shape of a hollow cylinder, one end of the support body (3) is connected to the fixing rod (1-5) by a pin, and the other end of the support body (3) is connected to the cutting electrode.

6. The single-piece uterine cavity electric resection device according to claim 5, wherein the cutting electrode is configured to be a needle electrode (7), one end of the cable (5) is connected to an extraction electrode arranged inside the fixing rod (1-5) or arranged on an outer surface of the fixing rod, a main body of the cable (5) is arranged inside the fixing rod (1-5), and the other end of the cable (5) is extended onto the support body (5).

7. The single-piece uterine cavity electric resection device according to claim 1, wherein the operating handle comprises a pair of handle bodies that are mutually hinged, the tensioning wire (1-6) is connected with one of the pair of handle bodies engaged on a corresponding end of the fixing rod (1-5), and the other one of the pair of handle bodies is connected with the traction wire (1-4).

8. The single-piece uterine cavity electric resection device according to claim 1, wherein a cross-sectional dimension of the fixing rod (1-5) is gradually decreased from one end of the fixing rod (1-5) provided with the operating handle to the other end of the fixing rod (1-5).

9. The single-piece uterine cavity electric resection device according to claim 1, wherein a swing angle of the support body (3) is ranged from 30 degree to 120 degree.

10. The single-piece uterine cavity electric resection device according to claim 1, wherein a diameter of the cutting electrode is ranged from 0.5 mm to 1.5 mm.
